(19) United States
(12) Patent Application Publication
(10) Pub. No.: US 2003/0091586 A1
(43) Pub. Date: May 15, 2003

(54) Peptides for inhibiting retroviruses

(76) Inventor: Mark E. Howard, Everett, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/255,733

(22) Filed: Sep. 25, 2002

Related U.S. Application Data
(63) Continuation of application No. 08/731,820, filed on Oct. 21, 1996.

(51) Int. Cl. C07H 21/04; A61K 39/21; A61K 39/12; C07K 2/00; C07K 4/00; C07K 5/00; C07K 7/00; C07K 14/00; C07K 16/00; C07K 17/00; A61K 38/00

(52) U.S. Cl. 424/187.1; 424/204.1; 536/23.72; 530/300

(57) ABSTRACT

Peptides derived from shark immunoglobulin preparations are used to prepare compositions, including pharmaceutical compositions, for inhibiting retrovirus replication in susceptible cells. The peptide preparations are useful for inhibiting diseases associated with retroviral infection, such as acquired immunodeficiency syndrome. The peptides also inhibit growth of tumor cells, especially sarcomas and leukemias.

### BACKGROUND OF THE INVENTION

The disease acquired immunodeficiency syndrome, or AIDS, remains substantially refractory to therapy. Despite intensive efforts to develop compounds that inhibit the virus that causes the disease, HIV, the infection almost uniformly progresses and the individual's immune system is rendered dysfunctional. Infected patients become extremely susceptible to secondary diseases, such as pneumonia and Kaposi's sarcoma, which are often life-threatening. While drugs such as 3′-azido-3′-deoxythymidine (AZT), 2′,3′-dideoxyinosine (ddI) and 2′,3′-dideoxycytosine (ddC) have been approved for use in infected individuals, profound toxicities and the emergence of drug-resistant viral strains are associated with their use. There remains a critical need to identify new anti-retroviral agents for use alone or in combination with other antiviral agents.

Effective anti-retroviral therapy depends on identifying antiviral agents devoid of significant toxicity and which, when employed in therapy, do not readily result in the emergence of drug-resistant viral isolates. Compounds potentially useful in inhibiting HIV and other retroviral infections are screened in a number of systems. Initially, screening is conducted in in vitro models of susceptible cell lines. Animal models are then used to identify those compounds with anti-retroviral activity in vivo as well as possessing acceptable levels of host toxicity. The models preferably assess activity against retroviral viremia as well as ability to suppress retroviral-induced disease, such as the destruction of the immune system and central nervous system disease in the case of HIV.

One model which has been found to be particularly useful as a model of retroviral infection, including HIV infection, is the Rauscher leukemia virus (RLV) infection of mice. RLV is infectious and pathogenic in adult mice, and it is erythrotropic, causing a splenomegaly that is proportional to viral titer. Chirigos, Cancer Res. 24: 1035-1041 (1964). Shortly after inoculation abnormal spleen colonies are formed whose numbers reflect viral titers. Each colony is the result of a successful viral “hit,” and the colony continues to enlarge while new target cells are being transformed continuously during the viremia. RLV also induces B-cell neoplasms. Thus, RLV infection results in a massive splenomegaly and erythroleukemia which kills infected animals within 4-5 weeks after inoculation. Weiss, Teich, Varmus, et al., RNA Tumor Viruses, 2d ed., Cold Spring Harbor Laboratory Press, pp 78-79, Cold Spring Harbor, N.Y. (1984). RLV infection in mice has also been shown to reproduce certain immunological aspects of HIV infection in humans. See, e.g., Gabrilovich et al., Immunology 82: 82-87 (1994) and Gabrilovich et al., Eur. J. Immunol. 23:2932-2938 (1993).

The use of the RLV model to identify and evaluate anti-HIV agents has become widespread. For example, RLV as a model of HIV infection has been reported for evaluating AZT (Ruprecht et al., Nature 323: 467-469 (1986); Ruprecht, Intervirol. 30(S1): 2-11 (1989)), new lipophilic derivatives of AZT (Schwendener et al., Antiviral Res. 24: 79-93 (1994)), derivatives of tetrahydroimidazole[4,5,1-jk][1,4]-benzodiazepin-2(1H)-thione (Buckheit et al., AIDS Res. Human Retrovir. 9: 1097-1106 (1993), biological response modifiers, including tests conducted by the U.S. Food and Drug Administration, as reported for, e.g., poly [I,C]-LC, MVE-2 and CL 246,738 (Black et al., Annl. N.Y. Acad. Sci. 685: 467-470 (1993)), and combination anti-HIV therapies (e.g., Ruprecht, supra, Buckheit et al., supra, and Black et al., supra). And, because RLV induces leukemia in infected animals, the RLV model is also used extensively as a model for treating various types of cancers, particularly leukemias. Sydow and Wunderlich, Cancer Lett. 82: 89-94 (1994).

New therapeutic modalities are urgently needed to provide more effective treatments for inhibiting retroviral infection, especially for HIV, and for treating the diseases associated with HIV infection. Also needed are effective means to inhibit development of cancers, such as leukemias or other neoplasms. Compositions useful for these purposes should be relatively easy to prepare and administer, relatively non-toxic, and effective inhibitors of retroviral infection or particular neoplasms. Quite surprisingly, the present invention addresses these and other related needs.

### SUMMARY OF THE INVENTION

The present invention provides, in one embodiment, a peptide composition, including pharmaceutical composition thereof, which comprises peptides derived from shark serum immunoglobulin. A peptide of the sequence Leu-Pro-Pro-Ser-Arg (SEQ ID NO: 1) is representative. The peptide composition can be formulated in amounts sufficient to inhibit retroviral replication in susceptible cells, or in amounts suitable for treating neoplastic disease, and further comprises a pharmaceutically acceptable carrier or stabilizer. Compositions of these peptides can be combined with other preparations for a synergistic anti-retroviral action. The peptide composition can also be supplied as a lyophilized preparation.

In other embodiments the invention provides methods for inhibiting retroviral infection of susceptible mammalian cells. An amount of peptide sufficient to inhibit or prevent said retroviral infection, is administered to and thereby contacted with the infected or infection-susceptible cells. The retrovirus susceptible to inhibition include the human retroviruses, including HIV-1 and HIV-2. The peptide, which can be prepared synthetically or by enzymatic digestion of shark immunoglobulin fractions, is administered in a variety of ways, including intravenously, topically, intramuscularly or orally.

In another aspect the invention provides methods for inhibiting tumor cells. A peptide preparation as described herein is administered to the tumor cells in an amount sufficient to inhibit growth of the tumor. The tumor cells can be in a culture, e.g., in vitro, in an afflicted mammal, or removed from the mammal for ex vivo treatment. The tumors susceptible to inhibition include a variety of sarcomas and leukemias, and further include those which are induced by a retroviral gene.

### DESCRIPTION OF THE SPECIFIC EMBODIMENTS

The present invention provides compositions useful in methods for inhibiting or reversing retroviral-mediated disease in an infected individual. It has been discovered as part of the present invention that preparations of peptides, including those prepared from digests of shark immunoglobulin molecules, inhibit manifestations of retroviral disease in an infected mammal. The peptide preparations are able to inhibit retroviral titer and associated symptoms and either restore cellular functions or prevent their further deterioration. In other aspects of the invention the peptide preparations are used to inhibit development of neoplastic disease, such as sarcomas or lymphomas, in afflicted mammals.

As part of the present invention it has been demonstrated that peptides can be prepared from shark immunoglobulin-containing fractions which possess significant anti-retroviral activity. The peptides, when administered to animals infected with the Rauscher murine leukemia virus, inhibit or prevent the development of splenomegaly in the animals in a dose dependent manner, whereas untreated animals develop severe splenomegaly. The Rauscher leukemia virus is a retrovirus widely used as an in vivo model of HIV retrovirus infection. A standard measure of drug effectiveness in the S Rauscher model has been the ability to inhibit splenomegaly in infected animals. In the experiments described herein the peptide preparations substantially prevent the splenomegaly observed in Rauscher infected control animals. These findings indicate that symptoms of retroviral disease, such as those which accompany HIV infection, can be inhibited or completely prevented, thereby allowing the immune system of the infected individual to respond more appropriately to other antigens for which an individual's response had been severely depressed, thus extending the life of the individual.

The peptide preparations described herein can be used to treat pathological conditions associated with the retroviral infection at the cellular level, such as HIV-induced neurological damage, retroviral-induced neoplastic diseases, programmed cell death, and the like. Retroviruses which can be treated by the peptide compositions include, for example, HTLV-I, HTLV-II, HIV-1, HIV-2, and a variety of animal retroviruses, as exemplified by the Rauscher murine leukemia virus. A disease condition amenable to treatment or inhibition by the peptide or immunoglobulins containing said peptide are identified using, e.g., mammalian cells or animals suspected of undergoing the disease state, e.g., sarcoma cells, which are confirmed to be susceptible to the process, e.g., tumorigenic in the case of the sarcoma cells. To determine that the peptide preparation inhibits the disease process, the cells are treated with the peptide preparation and the results compared to an untreated cell sample. In affected cells which have been treated and which demonstrate a inhibition or reversal of a conveniently monitored functional attribute(s), such as neoplastic proliferative capability, the process is determined to be susceptible to treatment according to the present invention. The peptides and methods of the present invention can also be used in treating autoimmune or autoimmune-associated diseases, particularly those which are associated with immunodeficiencies, as may be associated with HIV infection or the like.

In one embodiment a peptide is prepared synthetically or from shark serum immunoglobulin which has been fractionated, for example, as described in co-pending applications Ser. No. 60/005,133, Ser. No. 08/434,438, and PCT/US96/06245, each of which is incorporated herein by reference. Once the shark immunoglobulin is obtained it can be subjected to enzymatic digestion, preferably with papain or the like, to produce a cleavage product that yields a peptide useful in the present methods.

In embodiments of the invention the peptides contain from four to fifty amino acids of the sequence Leu-Pro-Pro-Ser-Arg (SEQ ID NO: 1) or the substantial equivalents thereof. Activity in the Rauscher assay, as well as immunoreactivty to antibodies produced to the peptide Leu-Pro-Pro-Ser-Arg (SEQ ID NO: 1), can be used to readily identify those which are substantially equivalent or which are immunologically competitive using these well known assay methods. Binding competition will typically be due to specific binding, but in some cases steric hindrance in conformation may also contribute to the competition.

In preferred embodiments described herein, the shark immunoglobulin peptides are derived from the sequence Leu-Pro-Pro-Ser-Arg (SEQ ID NO: 1) or are small molecule mimetics thereof. By “peptide” of the present invention is meant a contiguous chain of at least four amino acid residues from the sequence Leu-Pro-Pro-Ser-Arg (SEQ ID NO: 1) and usually will contain the five residues thereof, sometimes in a peptide of at least eight or nine, sometimes ten, eleven or twelve residues, and usually no more than about fifty residues, more usually fewer than about twenty-two, and preferably fewer than fifteen amino acid residues derived from said sequence or related sequences of other species. The term peptide is used in the present specification to designate a series of amino acids connected one to the other by peptide bonds between the alpha-amino and carboxy groups of adjacent amino acids. The peptides can be prepared “synthetically,” as described hereinbelow, or by recombinant DNA technology. The peptide will often be prepared substantially free of other naturally occurring immunoglobulins and fragments thereof. The peptide can be either in a neutral (uncharged) form or in a form which is a salt, and either free of modifications such as glycosylation, side chain oxidation, or phosphorylation or containing these modifications, subject to the condition that the modification not destroy the activity of the peptide as herein described.

Desirably, the peptide will be as small as possible while still maintaining substantially all of the antiretroviral activity of a larger peptide. By antiretroviral activity is meant the ability of a peptide of the invention to inhibit retroviral activity in vitro or in vivo, as occurs in a well accepted assay such as the Rauscher assay or the like.

A peptide of the invention can be optionally flanked and/or modified at one or both of the N- and C-termini, as desired, by amino acids from the immunoglobulin sequence, amino acids added to facilitate linking, delivery, labeling, other N- and C-terminal modifications, etc., as further described herein. The additional amino acids can be added to one or more termini of a peptide to provide for ease of linking peptides one to another, for coupling to a carrier, support or larger peptide, for modifying the physical or chemical properties of the peptide, etc. One or more amino acids such as tyrosine, cysteine, lysine, glutamic or aspartic acid, or the like, can be introduced at the C- or N-terminus of the peptide. In addition, a peptide sequence can differ from the native shark immunoglobulin sequence by being modified by amino terminal acylation, e.g., acetylation, or thioglycolic acid amidation, carboxy terminal amidation, e.g., ammonia, methylamine, etc. In some instances these modifications may provide sites for linking to a support or other molecule.

It will be understood that the peptides of the present invention or analogs or small molecule mimetics thereof which have antiretroviral activity may be modified as necessary to provide other desired attributes, e.g., improved antiretroviral activity or pharmacokinetic activity, while increasing or at least not significantly diminishing the activity of the unmodified peptide which is derived from the native immunoglobulin sequence. For instance, the peptides may be subject to various changes, such as insertions, deletions, and substitutions, either conservative or non-conservative, where such changes might provide for certain advantages in their use. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu; Asp, Glu; Asn, Gln; Ser, Thr; Lys, Arg; and Phe, Tyr. Usually, the portion of the sequence which is intended to substantially mimic an antiretroviral peptide will not differ by more than about 25% from the native sequence, except where additional amino acids may be added at either terminus for the purpose of modifying the physical or chemical properties of the peptide for, e.g., ease of linking or coupling, and the like. For the preparation of analogs of SEQ ID NO:1, a general method for site-specific incorporation of non-natural amino acids into proteins is described in Noren et al., Science 244:182-188 (1989), incorporated herein by reference.

By providing an abundant source of peptide of SEQ ID NO:1, the present invention enables quantitative structural determination of the peptide to design small molecule analogs and peptidomimetics of SEQ IDNO:1. The peptide sequence itself can be analyzed by a hydrophilicity analysis, e.g., Hopp et al., Proc. Natl. Acad. Sci. USA 78:3824 (1981), to identify regions of secondary structure. In addition, NMR, infrared, Raman, and ultraviolet analysis can be used to characterize the peptide and design mimetics of its structure. In particular, NMR provides a powerful structural analysis of molecules in solution which more closely approximates their native environment. Marion et al., Biochim. Biophys. Res. Comm. 113:967-974 (1983). Other methods can also be employed, including X-ray crystallography. Engstrom, Biochem. Exp. Biol. 11:7-13 (1974).

Various screening techniques are known in the art for screening for analogs of the peptides of the invention. Various libraries of chemicals and natural products are available. Identification and screening for analogs is further facilitated by determining structural features of the peptide as described above, to provide for the rational design or identification of analogs. Another approach uses recombinant bacteriophage to produce large libraries, e.g., as described in Scott et al., Science 249:386-390 (1990); Cwirla et al., Proc. Natl. Acad. Sci. USA 87: 6378-6382 (1990); Devlin et al., Science 249:404-406 (1990) for screening for mimetics. Another approach uses primarily chemical methods, as described in Geysen et al., J. Immunologic. Meth. 102:259-274 (1987), Fodor et al., Science 251: 767-773 (1991); Houghten (U.S. Pat. No. 4,631,211), Rutter (U.S. Pat. No. 5,010,175); Lam et al, WO 92/00252, and Blake (U.S. Pat. No. 5,565,325), each of the foregoing being incorporated herein by reference.

Having identified different peptides of the invention which are active against retroviruses, in some instances it may be desirable to join two or more peptides in a composition or admixture. The peptides in the composition can be identical or different, and together they should provide equivalent or greater activity than the parent peptide(s). For example, using the methods described herein, two or more peptides may define different or overlapping active sites from different or the same immunoglobulin region, which peptides can be combined in a cocktail to provide enhanced immunoreactivity.

The peptides of the invention can be combined via linkage to form polymers. Where the same peptide is linked to itself, thereby forming a homopolymer, a plurality of repeating units are presented. When the peptides differ, e.g., a cocktail representing different regions, heteropolymers with repeating units are provided. In addition to covalent linkages, noncovalent linkages capable of forming intermolecular and intrastructural bonds are also contemplated by the present invention.

Linkages for homo- or hetero-polymers or for coupling to carriers can be provided in a variety of ways. For example, cysteine residues can be added at both the amino- and carboxy-termini, where the peptides are covalently bonded via controlled oxidation of the cysteine residues. Also useful are a large number of heterobifunctional agents which generate a disulfide link at one functional group end and a peptide link at the other, including N-succidimidyl-3-(2-pyridyldithio) proprionate (SPDP). This reagent creates a disulfide linkage between itself and a cysteine residue in one protein and an amide linkage through the amino on a lysine or other free amino group in the other. A variety of such disulfide/amide forming agents are known. See, for example, Immun. Rev. 62:185 (1982). Other bifunctional coupling agents form a thioether rather than a disulfide linkage. Many of these thioether forming agents are commercially available and include reactive esters of 6-maleimidocaproic acid, 2 bromoacetic acid, 2-iodoacetic acid, 4-(N-maleimido-methyl) cyclohexane-1-carboxylic acid and the like. The carboxyl groups can be activated by combining them with succinimide or 1-hydroxy-2-nitro-4-sulfonic acid, sodium salt. A particularly preferred coupling agent is succinimidyl 4-(N-maleimidomethyl) cyclohexane-1-carboxylate (SMCC). Of course, it will be understood that linkage should not substantially interfere with the activity (e.g., antiretroviral or antitumor) of either of the linked groups.

As mentioned above, amino acid arms may be provided at the C- and/or N-terminus of the peptide or oligopeptide. If present, the arms will usually be at least one amino acid and may be 50 or more amino acids, more often 1 to 10 amino acids, and preferably less than 5 amino acids for ease of synthesis. The arms may serve a variety of purposes, such as spacers, to attach peptides to a carrier or delivery vehicle, etc. To provide useful functionalities for linking to a carrier, solid phase or to form higher-ordered structures, such as dimers, trimers, or other multimers, amino acids such as tyrosine, cysteine, aspartic acid, or the like, may be introduced at provided at the C- and/or N-terminus of the arm or peptide. To enhance active site presentation, of particular interest is the presence of from 1 to 10 amino acids at the C- and/or N-terminus, more preferably 1 to 5 amino acids, and most preferably about 1 to 3. Spacer residues between the peptide and a terminal functional group are typically Gly.

The peptides of the invention can be prepared in a wide variety of ways. Because of their relatively short size, the peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984); Tam et al., J. Am. Chem. Soc. 105:6442 (1983); Merrifield, Science 232:341-347 (1986); and Barany and Merrifield, The Peptides, Gross and Meienhofer, eds., Academic Press, New York, pp. 1-284 (1979), each of which is incorporated herein by reference. Short peptide sequences, or libraries of overlapping peptides, usually from about 6 up to about 35 to 50 amino acids, which correspond to the selected peptide region described herein, can be readily synthesized and then screened in screening assays designed to identify peptides having activity against retroviruses or tumors.

Alternatively, recombinant DNA technology may be employed wherein a nucleotide sequence which encodes a peptide of interest is inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1989); Ausubel et al., (ed.) Current Protocols in Molecular Biology, John Wiley and Sons, Inc., New York (1987), and U.S. Pat. Nos. 4,237,224, 4,273,875, 4,431,739, 4,363,877 and 4,428,941, for example, whose disclosures are incorporated herein by reference. Fusion proteins which comprise one or more peptide sequences of the invention can be used to present the peptide determinants of the invention.

As the coding sequence for peptides of the length contemplated herein can be synthesized by chemical techniques, for example, the phosphotriester method of Matteucci et al., J. Am. Chem. Soc. 103:3185 (1981), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences.

For preparing a pharmaceutically acceptable peptide composition, the peptide will typically be sterilized in a manner well known to those familiar with preparing pharmaceutically acceptable peptide preparations, e.g., by filtration, irradiation, etc.

The peptide compositions may be administered to persons or mammals suffering from, or predisposed to suffer from retroviral-associated disease or cancer. The peptide is believed to restore functionality, such as immunoproliferative capacity, etc., to HIV-afflicted cells. Thus, not only is replication or spread of the virus impeded by the treatment, but the patient regains, or retains, a responsive immune system and therefore is able to respond to other antigenic challenges and/or to HIV itself. As infections secondary to HIV are a major cause of morbidity, the treatment afforded by the present invention presents a major step toward eliminating the potentially devastating effects of this disease.

The compositions also find use for pre- or post-exposure prophylaxis, e.g., HIV prophylaxis following dirty needle injuries to health care workers or routinely accompanying blood-transfusions or to persons in danger of becoming exposed to infected body or culture fluids. For post-exposure prophylaxis, administration is begun shortly after the suspected inoculation and continues for at least about two to four weeks thereafter, followed by additional dosages or long term maintenance dosages as may be necessary to inhibit growth of the virus and disease and/or to maintain immunity thereto.

The pharmaceutical peptide compositions are intended for parenteral, topical, oral, or local administration for prophylactic and/or therapeutic treatment. Preferably, the pharmaceutical compositions are administered orally or parenterally, i.e., intravenously, intraperitoneally, subcutaneously, or intramuscularly. Thus, this invention provides methods which employ compositions for oral, topical or parenteral administration which comprise a solution of a peptide, separately or with substantially purified shark immunoglobulin and/or shark marrow, in a pharmaceutically acceptable carrier, preferably an aqueous carrier. A variety of aqueous carriers may be used, e.g., water, buffered water, 0.4% saline, 0.3% glycine, and the like, and may include other proteins for enhanced stability, such as albumin, lipoprotein, globulin, etc., subjected to mild chemical modifications or the like. The compositions of the invention can also be formulated into a cream or salve for topical or transdermal administration, e.g., at 5-25% concentration. The compositions may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as a pH adjusting and buffering agents, tonicity adjusting agents and the like, for example, sodium acetate, sodium lactate, sodium chloride, potassium chloride, calcium chloride, stabilizers (e.g., maltose (1-20%), etc.

The peptides of the invention may also be administered via liposomes. Liposomes, which include emulsions, foams, micelles, insoluble monolayers, phospholipid dispersions, lamellar layers and the like, can serve as a vehicle to target the peptides to a particular tissue, such as lymphoid tissue, retrovirally infected or tumor cells, as well as to increase the half-life of the peptide composition. In these preparations the peptide to be delivered is incorporated as part of a liposome, alone or in conjunction with a molecule which binds to, e.g., a receptor prevalent among lymphoid cells, tumor or retrovirally infected cells, such as monoclonal antibodies, or with other therapeutic or immunogenic compositions. A variety of methods are available for preparing liposomes, as described in, e.g., U.S. Pat. Nos. 4,837,028 and 5,019,369, incorporated herein by reference.

The concentration of the peptide, immunoglobulin preparation and/or marrow in these pharmaceutical formulations can vary widely, i.e., from less than about 10k, usually at or at least about 25% to as much as 75 or 90% by weight and will be selected primarily by fluid volumes, viscosities, etc., in accordance with the particular mode of administration selected and the disease being treated. Actual methods for preparing orally, topically and parenterally administrable compositions will be known or apparent to those skilled in the art and are described in detail in, for example, Remington's Pharmaceutical Science, 19th ed., Mack Publishing Company, Easton, Pa. (1995), which is incorporated herein by reference.

Determination of an effective amount of a composition of the invention to inhibit retroviral-mediated disease or cancer in a patient can be determined through standard empirical methods which are well known in the art. For example, reversal of impairment of immune function, e.g., restoration of lymphoproliferative response to recall antigen (e.g., influenza), alloantigens or mitogens such as PWM or PHA, and thus efficacy of the subject compositions, can be monitored with a variety of well known in vitro T-cell proliferative response procedures.

Compositions of the invention are administered to a host already suffering from a retroviral infection or neoplasm, as described above, in an amount sufficient to prevent or at least partially arrest the development of the ensuing immunodeficiency disease and its complications, or the susceptible tumor, as more fully described below. An amount adequate to accomplish this is defined as a “therapeutically effective dose.” Amounts effective for this use will vary considerably and depend on the severity of the infection or disease and the weight and general state of the patient being treated, but generally range from about 0.1 μg/kg to about 100 mg/kg host body weight of peptide, with dosages of from about 10 μg/kg to about 50 mg/kg per application being more commonly used. Administration is daily, weekly or less frequently, as necessary depending on the response to the disease and the patient's tolerance of the therapy. Maintenance dosages over a prolonged period of time may be needed, and dosages may be adjusted as necessary. The period of administration will generally be sufficient to restore the immune system of the host, such that effective immune responses can be mounted against a variety of antigens, most desirably the HIV virus in the case of individuals infected with HIV, or to eliminate or substantially inhibit the growth of the cancer cells. If an individual's restored immune system is not able to eliminate the disease, maintenance dosages over a prolonged period may be necessary. Also, it must be kept in mind that the materials of the present invention may be employed in life-threatening or potentially life threatening situations. In such cases, it is possible and may be felt desirable by the treating physician to administer substantial excesses of these compositions. In veterinary uses for treatment of other retroviral diseases or tumors, higher levels may be administered as necessary.

In prophylactic applications, compositions of the present invention are administered to a patient susceptible to or otherwise at risk of retroviral-mediated disease to enhance the patient's own immunologic capabilities. Such an amount is defined to be a “prophylactically effective dose.” In this use, the precise amounts again depend on the patient's state of health and weight, but generally range from about 0.1 μg/kg to about 75 mg/kg body weight, more commonly from about 1 μg/kg to about 50 mg/kg of body weight.

Single or multiple administrations of the compositions are carried out with the dose levels and pattern being selected by the treating physician. In any event, the pharmaceutical formulations of the peptide, separately or together with shark immunoglobulin and/or marrow should provide a quantity of inhibitor sufficient to effectively inhibit the retroviral-mediated disease or tumor in the afflicted host.

The methods of the invention can also be employed for ex vivo therapy. By ex vivo or extracorporeal therapy is meant that therapeutic manipulations are performed on host cells and fluids outside the body. For example, lymphocytes or other target cells may be removed from a patient and treated with high doses of the peptide composition, providing a concentration of inhibitor to the cell far in excess of levels which could be accomplished or tolerated by a patient. Following treatment, the cells are returned to the host to treat the disease.

For use in the present methods a preparation of a composition of the invention can be combined with one or more other pharmaceutical compositions for a variety of therapeutic uses, e.g., enhanced therapeutic activity against retroviruses such as HTLV-I, HTLV-II, HIV-1 or HIV-2, or cancer. For example, in the treatment of HIV infection, the pharmaceutical compositions of the present invention may be administered alone or as adjunct therapy with protease inhibitors, AZT, ddI, ddC, or combinations thereof, such as AZT, ddI, and peptide or shark immunoglobulin concentrate. The peptide can also be combined with the IgG-like fraction to achieve enhanced efficacy. The peptide can also be combined with shark marrow preparations. When administered as adjunct therapy, the preparations can be administered in conjunction with the other treatment modalities, or separately at different intervals.

The compositions of the present invention also find use in vitro. The compositions can be used to inhibit retroviral induced death of cultured cells, such as certain hybridoma or other lymphocyte lines which are susceptible to retroviral infection. The preparations of the invention can also be used in screening assays to assess effective levels of anti-retroviral compounds or other treatments. In addition, by determining whether a retrovirus-mediated dysfunction or death of a patient's cells is susceptible to inhibition or reversal by a composition of the invention, appropriate therapy can be more readily instituted or, alternatively, the effect of other treatment modalities, such as other anti-HIV regimens, can be determined. Thus, a diagnostic method for assessing the efficacy of, e.g., anti-HIV therapy is also provided by the present invention. Detecting changes in vitro regarding the level of HIV susceptibility, or restoration of immune function, e.g., response to recall antigens, to alloantigens, or to mitogens such as PWM or PHA, provides an indication of in vivo-activity of the peptide composition intended for treatment in accordance with the present invention.

To monitor changes in the level of immune function in a cell population, control values of immune function may be determined from cells from the general population or from the patient prior to commencement of therapy. Since immune function may vary considerably among patients, determination of each patient's pre-treatment immune function is preferred. The level of immune function in cells, e.g., lymphocytes in the case of HIV-infected individuals, is then monitored during therapy. This level is compared to the level of the immune function in cells not exposed to therapy, and effectiveness of therapy is assessed by an increased level in the measured immune function during or post-therapy.

The peptide compositions of the present invention can also be used as an anti-neoplastic agent. Among the neoplastic diseases targeted for inhibition by the peptides are sarcomas, leukemias, and carcinomas, including those which may be induced by a retroviral gene. Determination of an effective amount of peptide of the invention sufficient to inhibit growth of the neoplastic cells may be determined by, for example, monitoring metastatic sites with a variety of procedures, e.g., in vivo imaging or ex vivo diagnostic techniques. Other cancer markers may also be used to monitor therapy with the peptide compositions of the invention, e.g., the PSA assay for prostate carcinoma. The therapeutic compositions are administered to a patient already suffering from a neoplasm, e.g., sarcoma, leukemia or carcinoma, in an amount sufficient to cure or at least partially arrest the disease. An amount adequate to accomplish this is defined as “therapeutically effective dose.” Amounts effective for this use will depend on the severity of the neoplasm and its location, particularly when a metastatic site is implicated, and the weight and general state of the patient being treated, but generally range from about 1 μg/kg to about 100 mg/kg host body weight of peptide per day, with dosages of from about 10 μg/kg to about 75 mg/kg per day being more commonly used. Maintenance dosages over a prolonged period of time may be adjusted as necessary. As the peptide compositions may be employed in advanced disease states substantial excesses of these compositions may be administered.

Single or multiple administrations of the compositions can be carried out with the dose levels and pattern being selected by the treating physician. In any event, the pharmaceutical formulations should provide a quantity of peptide composition sufficient to effectively inhibit the neoplastic disease. The pharmaceutical compositions of the present invention may be administered alone or as adjunct therapy, e.g., with taxol, cis-platin, tamoxifen, etoposide phosphate, doxorubicin, daunomycin, endocrine therapy, etc. When administered as adjunct therapy, the compositions of the present invention may be administered in conjunction with the other treatment modalities, or separately at different intervals. The peptide preparations of the invention can also be used in ex vivo therapy of neoplastic disease. For example, bone marrow or other target cells or tissues are removed from a patient and treated with high doses of the peptide compositions, proving a therapeutic concentration far in excess of levels which could be accomplished or tolerated by the patient. Following treatment to eliminate the neoplastic cells in the target cell population or tissue, the cells or tissues are return to the patient.

The following experimental examples are offered by way of illustration, not by limitation.

### EXAMPLE I

This Example describes the preparation of shark immunoglobulin. Shark blood (whole blood with red cells) was received frozen. No attempt was been made to separate the serum from the cellular portion. A saturated ammonium sulfate solution was adjusted to pH 7.8 with 2N NaOH just prior to treating whole blood which had been brought to room temperature. The whole blood was diluted with an equal volume of saline (0.9% NaCl) solution to reduce its viscosity. With constant stirring 500 ml of ammonium sulfate solution was added dropwise to 1 liter of the diluted blood. At this point almost all of the hemoglobin and cellular matter were removed by precipitation. The material was centrifuged to remove cellular debris and hemoglobin. To the supernate another 500 ml of saturated ammonium sulfate was added and the suspension stirred for an additional 3 hours to avoid mechanical trapping of serum components other than gamma globulin in the precipitate.

The suspension was centrifuged at room temperature for 30 minutes at 1400× g (about 3000 RPM with a rotating radius of 14 cm). The first precipitate contained gamma globulin plus other globulins and traces of albumin. The precipitate collected in the centrifuge tubes was dissolved in enough saline to restore the volume of solution to the volume of the original sample. The gamma globulin fraction was purified by a second and third precipitation. To the 2 liter resuspended gamma globulin was added 500 ml saturated ammonium sulfate dropwise and stirred for 2 hours. The precipitate was recovered by centrifugation again and the step repeated.

After a third ammonium sulfate precipitation, the precipitate was dissolved in borate buffered saline (BBS) comprised of boric acid, 6.081 g; borax (sodium tetraborate, Na₂B₄O₇.10H2O), 9.536 g; sodium chloride, 4.384 g; and distilled water to 1 liter. If necessary, pH was adjusted with dilute HCl or NaOH solution to between 8.4 and 8.5. The BBS was prepared by mixing 5 parts of buffer with 95 parts saline.

The ammonium sulfate was removed from the suspension by dialyzing against BBS for several days at 4° C. The dialysate was changed early morning and night. The dialysate was checked daily for sulfate ions. The pH was checked to see that it stayed >7.0.

When the dialysis solution was free of sulfate ions the dialysate was removed from the dialysis bag. It was centrifuged at 3000 RPM at 4° C. for 30 minutes. The precipitate was collected. The yield from 1 liter of whole blood was 1.4 g of purified immunoglobulin.

To prepare the saturated ammonium sulfate solution, 1000 g (NH₄)₂SO₄ was heated with stirring in approximately 1 liter of water at 50° C. until most of the salt was dissolved. It was then allowed to stand overnight at room temperature. The desired pH was adjusted by the addition of 2N NaOH. For the precipitation of a serum sample at 0.5 (50%) saturation, 1 volume saturated ammonium sulfate solution was added to 1 volume of serum.

### EXAMPLE II

This Example describes the preparation of cleavage products of immunoglobulin isolated from shark blood.

Crystalline immunoglobulin prepared as described in Example I, believed to be IgG or an IgG-like molecule, was used to carry out the papain digestion. In a water bath at 37° C. 100 mg of dialyzed Ig in a reaction volume containing a final concentration of 0.002M EDTA, 0.001M cysteine and 1 mg of papain per 100 mg protein. The solvent for the reagents was 0.1M acetate, pH 5.5. A representative example comprised 5 ml protein solution (2% protein); 0.75 ml 0.02M EDTA in 0.1M acetate buffer, pH 5.5; 0.75 ml 0.01M cysteine in 0.1M acetate buffer, pH 5.5; 1.0 ml papain containing 1 mg enzyme/ml in 0.1M acetate buffer, pH 5.5.

The digestion was allowed to proceed for about 9 hours, at which time the digestion was considered to be complete. A sample of the Ig was processed as a control, but without the papain. Bacterial growth was inhibited by the addition of a few drops of toluene to the digestion mixture. The digestion was stopped by the addition of chloromercuriobenzoate to the final concentration of 0.001M. The digestion mixture was then dialyzed against two changes of 0.01M acetate buffer, pH 5.5.

To isolate the digested fractions, at room temperature a carboxymethylcellulose column approximately 1.5×50 cm was prepared using 0.O1M acetate buffer, pH 5.5. The sample was placed on the column, washing it with 2 ml amounts of starting buffer. After the sample was on the column, 150 ml of 0.01M acetate buffer pH 5.5 was added. Five milliliter fractions were collected. After all of the 0.O1M buffer had entered the column, 150 ml of each of the following buffers was added: 0.05M, 0.1M, 0.225M, and 0.45M acetate buffer, pH 5.5.

The peaks of protein eluted from the digestion mix were as follows: Fraction #1 (100 ml) 0.05M acetate buffer—FAB; Fraction #2 (250 ml) 0.1M acetate buffer—FAB; Fraction #3 (300 ml) 0.225M acetate buffer—FAB & Ig; Fraction #4 (400 ml) 0.45M acetate buffer—FC. The fractions were concentrated by per evaporation until a protein concentration of about 0.50 was obtained. At this point, the FC fraction could be filtered off.

The material precipitating was dissolved in 0.02N acetic acid and then extracted with ether (200 ml). The ether extracts were taken to a small volume (10 ml), and then held at about 5C, where crystallization of peptide occurred. In subsequent tests described below peptide having the sequence Leu-Pro-Pro-Ser-Arg (SEQ ID NO: 1) showed inhibitory activity against the Rauscher virus. Subsequently, the amino acids in the peptide were identified and synthesized.

### EXAMPLE III

This Example describes synthesis of peptide having the sequence Leu-Pro-Pro-Ser-Arg (SEQ ID NO: 1) identified in Example II.

For synthesis of Leu-Pro-Pro-Ser-Arg (SEQ ID NO:1), the following procedure is employed.

1. A solution of 5 g of Poly 1-leucine hydrobromide is dissolved in 200 ml of dimethylsulfoxide, 0.5 ml triethylamine is added. N-carboxy-L-Proline anhydride (14G prepared from L-proline and phosgene). It is used immediately after preparation since this anhydride is unstable. The L-proline is dissolved in 100 ml dimethyl sulfate. The two are added together and stirred vigorously at room temperature. Carbon dioxide evolution starts immediately and polymerization is allowed to proceed for 24 hours with stirring at room temperature.

2. The reaction mixture, which is opalescent and very viscous after 24 hours, is exhaustively dialyzed against distilled water to remove the dimethyl sulfoxide.

3. The gelatinous precipitate formed during the dialysis is concentrated by freeze drying and then dissolved in 300 ml of anhydrous formic acid at 25° C.

4. The resulting formic acid solution is kept for 1 hour at 25° C. and then dialyzed against several changes of distilled water.

5. The contents of the dialysis bags are concentrated by flash evaporation, lyophilized, and stored at −20° C.

6. 10 g of Leucine-Proline is then dissolved in 150 ml phosphate buffer (0.05 mol.) at pH 7.0 in a 1 liter flask. The flask is cooled with ice to 2° C. and a solution of 4.2 g of N-carboxy-γ benzyl L-Serine anhydride and 3.0 g N-carboxy-L-Arginine anhydride are dissolved separately in 120 ml of anhydrous dioxane. The two anhydrides are then mixed before the addition to the aqueous solution. The mixture is heated to 70° C. and stirred for 30 minutes. The precipitate is the active product.

7. To remove the protective groups. The above reaction mixture (4 g) is dissolved in 10 ml anhydrous trifluoroacetic acid, and then 30 ml of 45% HBr in glacial acetic acid is added. The mixture is kept at 4° C. for 72 hours. The final peptide Leucine-Proline-Proline-Serine-Arginine is isolated by precipitation into 500 ml of absolute ether. The precipitate is filtered and washed with additional cold ether. The crystalline product is dried over sodium hydroxide in vacuo.

In another aspect the peptide Leu-Pro-Pro-Ser-Arg (SEQ ID NO:1) is prepared as follows:

N-benzyloxycarboneal-o-tert. butyl-D-leucineamide (Z-D-Leu(Bu′-NH₂): Concentrated sulfuric acid (0.1 ml) and isobutylene (35 ml) are added to a suspension of N-benzyloxycarbonyl D-leucine (4.2 g) in methylene chloride (35 ml) kept in pressure resistant vessel cooled with dry ice/acetone. The reaction vessel is sealed and the temperature is allowed to rise to room temperature (20° C.). After 4 days, excess isobutylene is evaporated off and the organic solution is washed with aqueous 5% sodium carbonate (3×30 ml), 5% citric acid (20%) and then with water pH 6.0. The organic phase is dried over magnesium sulfate and concentrated to dryness yielding the above compound as a clear oil (4.60 g).

For preparation of [2-ethoxy-carbonyl-6-test-butoxy-carbonyl-amino]hexanolyl-L-Proline benzyl ester (Leu-Pro-Pro): The previous leucine derivative (0.6 g, 0.98 mmol) is dissolved in CH₂C1₂ (3 ml) added to the mixture. The reaction mixture is cooled to 0° C. and a solution of DCCI (decyclohexylcarbodamide) (0.495 g 2.4 mmol) in CH₂Cl₂ is added at this temperature. The reaction mixture is stirred at 0° C. for 30 minutes and then at room temperature for an additional 20 minutes. The activated ester thus prepared is filtered into a reaction flask containing a solution of Proline benzyl ester hydrochloride (0.532 g, 2.2 mmol) in CH₂C1₂ (60 ml) to which is added NMM (N methylmorpholine) (0.242 ml, 2.2 mmol), necessary to remove the hydrochloride previously added. The mixture is stirred at room temperature overnight, the solvent is then evaporated off and the oily residue is taken up in small amounts of ethyl acetate and kept at −25° C. for 1 hour.

The precipitate is removed by filtration and the filtrate is diluted with an additional amount of ethyl acetate. The ethyl acetate solution is washed with 5% aqueous NaHCO3 (5×100 ml) with 5% aqueous citric acid (3×50 ml) and finally with deionized water, pH 6.0-6.5. The organic phase is dried over anhydrous magnesium sulfate and then concentrated to dryness under vacuum. The compound prepared is OET-(R.S.) leucine (N-Boc)-L-Pro-OH. This is a yellow oil.

The second proline group is put on by repeat of the above sequence of step making the Leu-Pro-Pro by the above steps.

For preparation of Leu-Pro-Pro-Ser: The protected OET (R.S.) Leu (N-Boc)-L Pro-L Pro (MTR)-OBu′) is mixed in a solution of HOBT (N hydroxybenzotriazole) (0.261 g, 1.93 mmol) in CH₂CL₂ (3 ml) and DMF (dimethylformamide) (0.5 ml) is added to BOC-SER (B21)-O-Resin (10.1) 2.1 mmol) in 50 ml CH₂Cl₂. The mixture is cooled to 0° C. and DCCI (0.398 g, 1.93 mmol) is added. The reaction is then stirred at 0° C. for 30 minutes and then at room temperature for another 30 minutes. The three activated ester is filtered into a reaction flask containing a solution of the compound. When the reaction is over, a small amount of ethyl acetate is added to take up the product and cooled to −25° C. for one hour. The precipitate is removed by filtration, the filtrate is diluted with 100 ml additional ethyl acetate and washed with a 5% NaHCO₃ aqueous solution (4×100 ml), with a saturated sodium chloride solution and finally with water pH 6.0-6.5. The organic solution is dried over MgSO₄ and the solvent evaporated under vacuum yielding the desired compound as a yellow oil.

For preparation of Leu-Pro-Pro-Ser-Arg ((HO-RgS) leucine (NBOC) L-Pro-L Pro L Ser-LArg (NG*MTR)-OBu′)) (MTR=(2,3,6 trimethyl-4-methoxyphenyl) sulphonyl): 1M KOH in absolute ethanol (2 ml, 2 mmol) is added to a stirred solution of the compound obtained in the previous step. (0.857 g, 10.4 mmol) in absolute alcohol (10 ml) cooled to 0° C., and the reaction mixture is stirred overnight. The mixture is then diluted with 10 ml water. The ethanol is evaporated off and the pH is adjusted to 3 by the addition of citric acid. The acidic mixture is then extracted with ethyl acetate (4×60 ml), the organic phase is then combined and washed with saturated aqueous sodium chloride solution, and then with water to bring the pH to 7.0. The organic phase is dried over anhydrous magnesium sulfate. The solvent is then evaporated off yielding the peptide.

The compound obtained in the previous step is dissolved in TFA (tetrahydrofuran) (20 ml) containing 6% thioanisole. The solution is stirred for 4 hours. The TFA and thioanisole are then allowed to evaporate in a stream of nitrogen throughout the mixture. The residue is then taken up in methy cyanide and then concentrated to dryness under vacuum. The residue is then taken up in a few drops of methyl cyanide. It is then washed with ether (2×30 ml). The ether is then evaporated off and the aqueous phase is then freeze dried.

Thus is obtained a pure compound having the structure leucine-proline-proline-serine-arginine (SEQ ID NO:1).

### EXAMPLE IV

This Example describes the use of the peptide Leu-Pro-Pro-Ser-Arg (SEQ ID NO: 1) described above and shark immunoglobulin to inhibit manifestations of retrovirus infection in a mammal.

Efficacy of the peptide and shark Ig as anti-retroviral agents was determined using the murine leukemia virus (MuLV) (also known as the Rauscher virus) disease model. Rauscher is a pathogenic murine retrovirus in mice, typically causing erythroid colonies in the spleen of mice leading to a severe splenomegaly, and also causes a erythroleukemia. In this study, BALB/c mice treated with the peptide or shark Ig preparation and untreated controls were infected with virus preparations. The study was conducted as generally described in co-pending applications Serial Nos. 60/005,133, 08/434,438, and PCT/US96/06245, each incorporated herein by reference.

The Murine leukemia virus (MuLV) (Rauscher) was purchased from advanced Biotechnologies, Inc., Columbia Md. 21046, Lot No. 1/29/74. It was cryopreserved in JLS-V9/MuLV cells at 1.16×10⁷ cells/ml, with 90% viability.

The virus was suspended in RPMI 1640, with 10% fetal bovine serum, 50 μg/ml gentamicin, and 10% DMSO. The virus particle count was 5.57×1010 virus particles per ml. The reverse transcriptase activity was 97.8%. The virus was stored at −70 to −80° C.

Percent inhibition of viral activity was calculated based on the spleen size according to the following equation:

$$\% Inhib . = \frac{\begin{matrix}( mean spleen wt controls - \\ mean spleen tumor size treated )\end{matrix}}{( mean tumor size control )} \times 100$$

The Balb/C mice, 3-4 weeks old were used in the following experiments. All mice that received murine leukemia virus (MuLV) Rauscher were given the virus at 2.0×10⁶ virus particles intraperitoneally. Treatment was by intraperitoneal injection of the test material except in the case of the mice treated orally. Test material was administered at day 2, 4, and day 6 after tumor virus transfer.

Table I presents the data obtained from a number fractions obtained from the fractionation of shark blood Ig together with the activity of the synthetic peptide (SEQ ID NO:1).

TABLE I
Summary of Rauscher Leukemia Virus (MuLV) Challenge

| | No. | Mean Body | Mean Spleen | % |
|---|---|---|---|---|
| Group | Mice | Wt.g. | Wt. mg. | Inhibition |
| I. Control, No Treatment | 6 | 22.3 | 93.9 | — |
| No MuLV Rauscher | | | | |
| II. No Treatment | 6 | 22.3 | 4,183 | — |
| MuLV Rauscher | | | | |
| III. 1 mcg/kg Peptide (SEQ ID NO:1) | 7 | 24.5 | 614.2 | 85.4 |
| I.P. 3 doses | | | | |
| IV. 10 mcg/kg Peptide (SEQ ID NO:1) | 7 | 23.2 | 96.5 | 97.3 |
| I.P. 3 doses | | | | |
| V. 20 mcg/kg Peptide (SEQ ID NO:1) | 7 | 22.9 | 96.1 | 97.7 |
| I.P. 3 doses | | | | |
| VI. Shark α-2 Macroglobulin | 7 | 26.2 | 104.2 | 97.5 |
| 10 mcg, I.P. 3 doses | | | | |
| VII. 20 mcg, Shark IgG, | 6 | 26.2 | 245.1 | 61.7 |
| 3 doses | | | | |
| VIII. 20 mcg, FAB | 6 | 25.2 | 2,340 | 53.6 |
| IX. 20 mcg, FC | 6 | 24.8 | 149.5 | 96.5 |

Peptide (SEQ ID NO:1) gave an inhibition of splenomegaly at 1 mcg of the peptide given orally of 85.4. At 10 and 20 mcg doses, given 3 times, the inhibition was 97.3 and 97.7. This is substantially equivalent to achieving 100% inhibition in this assay. This inhibition was comparable to 20 mcg of FC fraction from which the original peptide was isolated. The shark IgG-like molecule at 20 mcg gave a 53.6% inhibition in this study.

Another significant finding was that shark blood contains a specific α-2 macroglobulin which under the same conditions at 10 mcg/kg body weight gave a 97.5% inhibition of spleen weight increase. The extraction of the a-2 macroglobulin is different than that for the shark blood IgG.

The next study compared the efficacy of oral versus injection administration of peptide and immunoglobulin. The study was performed as described above, and test material was administered on Days 2, 4, and 6. The results are shown in Table II.

TABLE II
Rauscher Test - Comparison of Oral Dosing Versus Injection.

| | No. | Mean Body | Mean Spleen | % |
|---|---|---|---|---|
| Group | Mice | Wt.g. | Wt. mg. | Inhibition |
| I. Control, No Treatment, | 5 | 25.9 | 100.8 | — |
| No MuLV Rauscher | | | | |
| II. Control - No Treatment | 5 | 30.1 | 3,630 | — |
| MuLV Rauscher | | | | |
| III. Oral 11 mcg/kg Peptide | 5 | 28.5 | 2,048 | 43.6 |
| MuLV Rauscher | | | | |
| IV. Oral 20 mcg/kg Peptide | 5 | 29.2 | 1,370 | 62.3 |
| MuLV Rauscher | | | | |
| V. Oral 40 mcg/kg Peptide | 5 | 25.6 | 945.4 | 74.0 |
| MuLV Rauscher | | | | |
| VI. Oral Shark Ig, 100 mcg | 5 | 27.9 | 975.0 | 73.1 |
| VII. Oral Shark Ig, 50 mcg | 5 | 24.1 | 1734 | 57.2 |
| VIII. Shark Blood, 0.5 ml, Oral | 4 | 24.4 | 1792.5 | 60.6 |
| f | | | | |
| IX. 10 mcg Peptide, I.P. | 6 | 26.5 | 106.8 | 97.1 |

Table II tabulates the results of dosing the mice with Rauscher MuLV by the oral route. In this test, 10 mcg/kg of the peptide given orally on day 2, 4, and 6 gave 43.6k inhibition. At 20 mcg/kg body weight the inhibition of this viral tumor model was 62.3%. At 40 mcg/kg body weight the inhibition was 74.0%.

The orally administered shark immunoglobulin at 100 mcg/kg body weight gave an inhibition of 73.1%. At 50 mcg/kg body weight the inhibition was 57.2%. This compares with 60.6% in mice given 0.5 ml of whole shark blood three times.

The peptide SEQ ID NO:1 given by injection at 10 mcg/kg body weight gave 97.1k inhibition.

In another study the efficacy of different concentrations of peptide and route of administration were compared. The study was performed as described above, and test peptide was administered on Days 1, 3, 5, 7, and 11. The results are shown in Table III.

TABLE III
Rauscher Leukemia Virus Test - Peptide Inhibition

| | No. | Mean Body | Mean Spleen | % |
|---|---|---|---|---|
| Group | Mice | Wt.g. | Wt. mg. | Inhibition |
| I. Control, - No Treatment, | 12 | 23.4 | 96.1 | — |
| No MuLV Rauscher | | | | |
| II. No Treatment | 12 | 23.0 | 2566.5 | — |
| MuLV Rauscher | | | | |
| III. 20 mcg/kg Peptide SEQ ID NO:1 | 12 | 22.5 | 119.8 | 95.34 |
| I.P., Days 1,3,5,7,11 | | | | |
| IV. 10 mcg/kg Peptide SEQ ID NO:1 | 12 | 22.2 | 1057.9 | 58.8 |
| I.P., Days, 1,3,5,7,11 | | | | |
| V. 20 mcg/kg Peptide SEQ ID NO:1 | 12 | 22.4 | 1440.0 | 43.9 |
| Orally, Days 1,3,5,7,11 | | | | |

Table III Summarizes the results of inhibition of spleen size in mice infected with Rauscher Murine leukemia virus. In this study 20 mcg/kg of the peptide SEQ ID NO:1 given intraperitoneally on days 1, 3, 5, 7, and 11 gave almost complete inhibition. At 10 mcg/kg the peptide given in 5 doses I.P. gave an inhibition of 58.8%. The peptide given on days 1, 3, 5, 7, and 11 gave an inhibition of 43.9% over the non-treated controls.

These studies demonstrate that peptide SEQ ID NO:1 and shark blood possess a powerful inhibitor of spleen enlargement (splenomegaly) in the Rauscher Mouse leukemia virus model. In this model inhibition of the virus correlates to inhibiting or interfering with reverse transcriptase, an enzyme necessary for virus growth.

In general, there is agreement between the various groups and different animal trials. The peptide SEQ ID NO:1 given intraperitoneally at 10 mcg or orally at 100 mcg/kg had a significant effect on inhibiting the spleen growth under these conditions. The results also confirm that the shark Ig preparation contained significant anti-retroviral activity and effectively prevented, in the case of the 0.5 ml treatment, or inhibited in the case of the lower doses, the normal progression of retroviral disease in the infected animals.

From the foregoing it will be appreciated that, although specific embodiments of the invention have been described herein for purposes of illustration, various modifications may be made without deviating from the spirit and scope of the invention. Accordingly, the invention is not to be limited except as by the appended claims.

What is claimed is:

1. A peptide of five to fifteen amino acids comprising Leu-Pro-Pro-Ser-Arg (SEQ ID NO: 1) which inhibits retroviral mediated disease.

2. The peptide of claim 1, comprising five to ten amino acids which comprise Leu-Pro-Pro-Ser-Arg (SEQ ID NO: 1).

3. The peptide of claim 2, which is five to nine amino acids which comprise Leu-Pro-Pro-Ser-Arg (SEQ ID NO: 1).

4. The peptide of claim 3, which is Leu-Pro-Pro-Ser-Arg (SEQ ID NO: 1).

5. A pharmaceutical composition which comprises a peptide of five to fifteen amino acids comprising Leu-Pro-Pro-Ser-Arg (SEQ ID NO: 1) which inhibits retroviral mediated disease and a physiologically acceptable carrier.

6. The pharmaceutical composition of claim 5, wherein the peptide is Leu-Pro-Pro-Ser-Arg (SEQ ID NO: 1).

7. The pharmaceutical composition of claim 5, further comprising an shark serum concentrate or immunoglobulin purified from a shark.

8. The pharmaceutical composition of claim 5, further comprising shark marrow.

9. A method for inhibiting retroviral infection of susceptible mammalian cells, which comprises administering to the cells a peptide according to claim 1 in an amount sufficient to inhibit said infection.

10. The method of claim 9, wherein the retrovirus is a human retrovirus.

11. The method of claim 10, wherein the human retrovirus is HIV-1.

12. The method of claim 9, wherein the peptide is administered to a mammal to inhibit retroviral replication in said mammalian cells.

13. The method of claim 12, wherein the peptide is administered intravenously or intramuscularly.

14. The method of claim 9, wherein the peptide is five to nine amino acids which comprise Leu-Pro-Pro-Ser-Arg (SEQ ID NO: 1).

15. The method of claim 14, wherein the peptide is Leu-Pro-Pro-Ser-Arg (SEQ ID NO: 1).

16. A method for inhibiting tumor cells, which comprises administering to the tumor cells a peptide according to claim 1 in an amount sufficient to inhibit growth of said tumor cells.

17. The method of claim 16, wherein the peptide is five to nine amino acids which comprise Leu-Pro-Pro-Ser-Arg (SEQ ID NO: 1).

18. The method of claim 17, wherein the peptide is Leu-Pro-Pro-Ser-Arg (SEQ ID NO: 1).

19. The method of claim 16 for inhibiting tumor cells, wherein the tumor cells are contained in an afflicted mammal and the peptide is administered to said mammal.

20. The method of claim 19, wherein the tumor is induced by a retroviral gene.


SEQUENCE LISTING (1 sequence)

SEQ ID NO: 1 - protein, 5 aa
LPPSR